(19) United States
(12) Patent Application Publication
(10) Pub. No.: US 2021/0261488 A1
(43) Pub. Date: Aug. 26, 2021

(54) CATALYTIC ETHYNYLATION

(71) Applicant: DSM IP ASSETS B.V., Heerlen (NL)

(72) Inventors: Fabrice AQUINO, Kaiseraugst (CH); Werner BONRATH, Kaiseraugst (CH); Francesco PACE, Kaiseraugst (CH); Peter RUCKSTUHL, Kaiseraugst (CH); Konrad WITZGALL, Kaiseraugst (CH)

(21) Appl. No.: 17/264,187

(22) PCT Filed: Jul. 29, 2019
(86) PCT No.: PCT/EP2019/070295

§ 371 Date: Jan. 28, 2021

(30) Foreign Application Priority Data
Jul. 30, 2018 (EP) 18186350.7

(51) Int. Cl. C07C 29/42; B01J 23/04

(52) U.S. Cl. CPC C07C 29/42; C07C 403/08; B01J 23/04

(57) ABSTRACT

The present invention relates to the catalytic ethynlation of αβ-unsaturated ketones for producing tertiary acetylenic alcohols.

The present invention relates to the catalytic ethynylation of α,β-unsaturated ketones for producing tertiary acetylenic alcohols.

The ethynyated reaction products, α-alkynols, are important intermediates in organic synthesis. For example, ethynyl-β-ionol is an intermediate used in the preparation of vitamin A and β-carotene.

The process for ethynylating of α,β-unsaturated ketones for producing tertiary acetylenic alcohols is well known and described in many patent (i.e. in U.S. Pat. Nos. 3,709,946; 3,082,260, 3,283,014, 4,147,886 and 4,320,236).

In U.S. Pat. No. 4,320,236, the ethynylation is carried out in the presence of a monolithium acetylide-ammonia complex.

In U.S. Pat. No. 4,147,886, the ethynylation is carried out in the presence of diluted KOH.

Due to the importance of the ethynylated reaction products, there is always a need for an improved way to producing such products.

Surprisingly, it was found that the use of solid KOH in the reaction process allows to carry out the ethynylation process in an easy way.

Therefore, the present invention relates to a process (P) to produce compounds of formula (III)

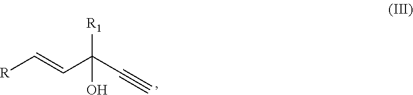

wherein

R is hydrogen or an aliphatic, cyclo-aliphatic or aromatic hydrocarbon which can be unsubstituted or substituted with lower alkoxy or lower alkyl groups and

R₁ is CH₃ or CH₂CH₃, (α-alkynol),

wherein a compound of formula (I)

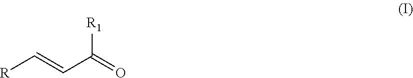

wherein the substituents have the same meanings as defined above

is reacted with a compound of formula (II)

≡ (II)

in NH₃ as a solvent and in the presence of KOH,

characterized in that KOH is added to reaction mixture in solid form.

Surprisingly the process is working without the addition of an aqueous solution of KOH.

The new process, which is characterized that the catalyst (KOH) is added in pure (=solid) form has some advantages in view of the prior art processes.

The reaction is carried out with water or with a small amount of water, wherein the water is added at the end of the reaction. This is very advantageous for the further work up of the reaction product at the end of the process.

Furthermore, the process is very easy to handle. The KOH is added to the reaction mixture in its pure form. No dilution step for KOH is needed. This step is not needed anymore.

The process according to the present invention is as disclosed above the following:

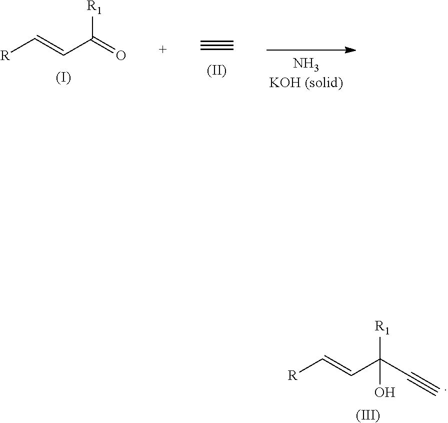

wherein R and R₁ are as defined above.

In a preferred embodiment of the present invention compounds of formula (III) are produced wherein

R is an aliphatic, cyclo-aliphatic or aromatic hydrocarbon which is substituted with lower alkoxy or lower alkyl groups and

R₁ is CH₃.

Therefore, the present invention also relates to a process (P1), which is process (P), wherein the compound of formula (I) and (III) R is an aliphatic or aromatic hydrocarbon which is substituted with lower alkoxy or lower alkyl groups and R₁ is CH₃.

An especially preferred process according to the present invention relates to the process of production of the compound of formula (III′)

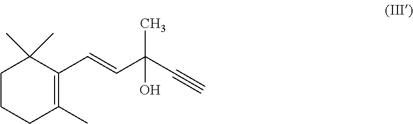 [SMILES: C#CC(C)(O)/C=C/C1=C(C)CCCC1(C)C]

by using the compound of formula (I′)

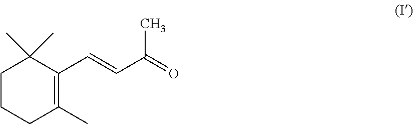

as starting material.

Therefore, the present invention also relates to a process (P2), which is process (P) or (P1), wherein the compound of formula (I) is the compound of formula (I′)

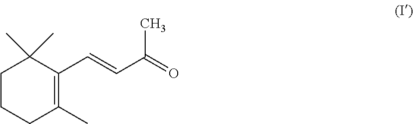

and the compound of formula (III) is the compound of formula (III′)

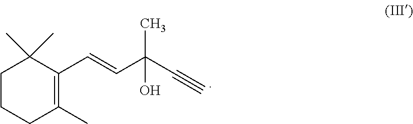 [SMILES: C#CC(C)(O)/C=C/C1=C(C)CCCC1(C)C]

The process according to the present invention is carried at low temperature. Usually the process is carried out at a temperature below +5° C. Preferably the process is carried out at a temperature of from −60° C. to 5° C.

Therefore, the present invention also relates to a process (P3), which is process (P), (P1) or (P2), wherein the process is carried out at a temperature below +5° C.

Therefore, the present invention also relates to a process (P4), which is process (P), (P1), (P2) or (P3), wherein the process is carried out at a temperature of from −60° C. to −5° C.

The reaction time of the process of the present invention is usually in the range of 0.5 to several hours, Usually the process is carried out during 0.5-12 hours.

Therefore, the present invention also relates to a process (P5), which is process (P), (P1), (P2), (P3) or (P4), wherein the process is carried out during 0.5-12 hours.

The process of the present invention is carried out in the presence of ethyne (compound of formula (II)). Ethyne is also known as acetylene. Ethyne is a colourless gas. The reaction according to the present invention can be carried out at atmospheric condition by adding ethyne to the reaction mixture or it can be done at elevated pressure (usually 2-10 bar) in a pressure resistant vessel.

Therefore, the present invention also relates to a process (P6), which is process (P), (P1), (P2), (P3), (P4) or (P5), wherein the process is carried out at atmospheric conditions.

Therefore, the present invention also relates to a process (P7), which is process (P), (P1), (P2), (P3), (P4) or (P5), wherein the process is carried out at elevated pressure (usually 2-10 bar).

As disclosed above the reaction according to the present invention is carried out without or with a small amount of water, wherein the water is added to the reaction mixture at the end of the reaction.

By the term “without any water” is meant that no water is added to the process intentionally. It might be that for example any used material of the process can comprise traces of water.

When water is used in the process according to the invention it is added at the end of the reaction (usually before the work up of the reaction mixture will be carried out).

Therefore, the present invention also relates to a process (P8), which is process (P), (P1), (P2), (P3), (P4), (P5), (P6) or (P7), wherein the process is carried without any water.

Therefore, the present invention also relates to a process (P9), which is process (P), (P1), (P2), (P3), (P4), (P5), (P6) or (P7), wherein water is added to the reaction mixture at the end of the reaction process.

When water is added it is added in a small amount. Usually not more than 50 mol equivalent (in regard to mol of the compound of formula (I)). Usually (and preferred) between 1-50 mol equivalent. More preferred 2-20 mol equivalent (in regard to mol of the compound of formula (I)).

Therefore, the present invention also relates to a process (P9′), which is process (P9), wherein less than 50 mol equivalent (in regard to mol of the compound of formula (I)) of water is added.

Therefore, the present invention also relates to a process (P9″), which is process (P9), wherein between 1-50 mol equivalent (in regard to mol of the compound of formula (I)) of water is added.

Therefore, the present invention also relates to a process (P9″), which is process (P9), wherein between 2-20 mol equivalent (in regard to mol of the compound of formula (I)) of water is added.

The reaction product (compound of formula (III)) can be removed from the reaction mixture by commonly known processes. Usually it is done by extraction.

The yields, conversions and selectivity of the process according to the present invention are excellent.

The compounds, which are produced by the process according to the present invention (compounds of formula (III)) are usually (and preferably) used in the manufacture of vitamin A or β-carotene.

The following examples illustrate the invention.

All % are related to weight and the temperature is given in ° C.

### EXAMPLES

### Example 1

66 mMol β-ionone were added to a mixture of 16 mMol KOH and 23 w % C₂H₂ in NH₃ and agitated for 1 h, at −10° C. at a pressure of 4.2 bar.

Afterwards 200 ml n-hexane were added slowly to the reaction mixture and the reaction mixture was degassed for about 2 h.

The inorganic phase was separated from the organic phase, neutralized with AcOH and extracted with 150 ml n-hexane.

The combined organic phases were washed with 150 ml water, dried on Na₂SO₄, filtered and concentrated under vacuum.

The yield was 77%.

### Example 2

The same reaction conditions as in Example 1 was chosen for Example 2. In addition, 10 ml water were added after 1 hour.

The yield was 81%.

1. A process for the production of compounds of formula (III) 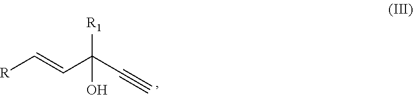
wherein
R is hydrogen or an aliphatic, cyclo-aliphatic or aromatic hydrocarbon which can be unsubstituted or substituted with lower alkoxy or lower alkyl groups and
R₁ is CH₃ or CH₂CH₃,
wherein a compound of formula (I) 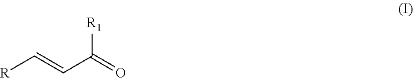
wherein the substituents have the same meanings as defined for the compound of formula (III)
is reacted with a compound of formula (II)
≡ (II)
in NH₃ as a solvent and in the presence of KOH,
characterized in that KOH is added to reaction mixture in solid form, and
wherein the process is carried without any addition of water.

2. Process according to claim 1, wherein
R is an aliphatic or aromatic hydrocarbon which is substituted with lower alkoxy or lower alkyl groups and
R₁ is CH₃.

3. Process according to claim 1, wherein the compound of formula (I) is the compound of formula (I′) 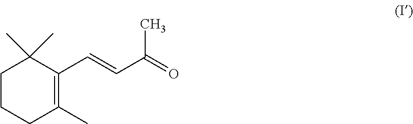
and the compound of formula (III) is the compound of formula (III′)

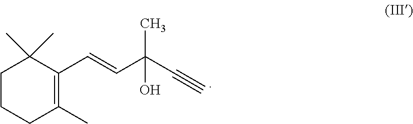 [SMILES: C#CC(C)(O)/C=C/C1=C(C)CCCC1(C)C]

4. Process according to claim 1, wherein the process is carried out at a temperature below +5° C.

5. Process according to claim 1, wherein the process is carried out at a temperature of from −60° C. to 5° C.

6. Process according to claim 1, wherein the process is carried out at atmospheric conditions.

7. Process according to claim 1, wherein the process is carried out at elevated pressure.

8. Process according to claim 1, herein the process is carried without any addition of water.
